(19) United States
(12) Patent Application Publication
(10) Pub. No.: US 2005/0027237 A1
(43) Pub. Date: Feb. 3, 2005

(54) Apparatus and methods for control of intravenous fluids

(76) Inventor: Dan Weiner, New York, NY (US)

Correspondence Address: Peter L. Berger, Esq.;Levisohn, Berger & Langsam, LLP, 19th Floor, 805 Third Avenue, New York, NY, 10022, US

(21) Appl. No.: 10/896,467

(22) Filed: Jul. 22, 2004

Related U.S. Application Data
(63) Continuation-in-part of application No. 09/702,212, filed on Oct. 31, 2000, now abandoned.
Application No. 09/702,212 is a division of application No. 08/922,887, filed on Sep. 3, 1997, now Pat. No. 6,165,151.
(60) Provisional application No. 60/488,957, filed on Jul. 22, 2003.

(51) Int. Cl. A61M 31/00

(52) U.S. Cl. 604/66

(57) ABSTRACT

A system for automatically monitoring the condition of a plurality of trauma patients by deriving real time blood pressure levels and automatically controlling the fluid flow in a gravity fed system into patients by adjustable clamps which are mechanically controlled by a micromotor to bear on the IV lumen, thereby controlling its cross-sectional area to so adjust fluid flow to the patient. A patient's desired blood pressure level may be maintained in order to reduce severe medical problems when the patient is in trauma. The housing for the system is a two piece hingedly connected assembly which includes a longitudinal channel which hugs the IV lumen when the two pieces are closed together in the manner of a book.

### RELATED APPLICATIONS

The present application is a non-provisional patent application based on provisional patent application Ser. No. 60/488,957, filed in the United States on Jul. 22, 2003, which itself was a continuation-in-part of Ser. No. 09/702,212, filed on Oct. 31, 2000, which is a continuation of Ser. No. 08/922,887, filed on Sep. 3, 1997, which issued as U.S. Pat. No. 6,165,151 on Dec. 26, 2000; the contents of the above related applications are fully incorporated herein by reference.

### DESCRIPTION OF THE INVENTION

The invention relates to the automatically controlled administration of intravenous fluids and or drugs to patients, thereby eliminating the need for continuous supervision and the monitoring of those patients while they are receiving their I.V. therapy.

This application is a continuation in part of my prior US Patent application identified above which describe a control system that functions utilizing a servo system to mechanically control the size of the cross-sectional area of an IV tube lumen, thereby controlling the rate of flow through that IV tubing to the patient. The mechanical compression of an adjustable clamp is driven by a micro-motor which responds in real time to physiologic data it receives from a patient monitor. This is a closed loop servo system which can be set to maintain pre-determined values; has safeguard emergency cut offs; and although intermittent oversight is desirable, it does not require continuous monitoring or supervision to be effective and safe. Additionally, a closed loop servo system operates to smooth out differences between real time blood pressure and a desired level so that reaching such level is smoothly achieved.

The simplicity of gravity flow; the low power needs of the unit which can adequately be supplied by batteries; the many therapeutic applications of this device, particularly in emergency clinical situations; the built in safety features; the portability and external shock resistance of the unit; the closed loop automated features that permit non-supervised use; its ability to be a medical care giver “extender” in mass casualty settings all enable a single medical professional to safely and effectively control the concurrent treatment of multiple patients. All of these features and capabilities serve to make this device very important with multiple applications in non-hospital environments. This would include military field or combat medicine or any mass casualty situation, civilian or military.

An important aspect of the present invention is the unique compact arrangement of the entirety of the device. In particular, the device has a book-like shape with a hinge about which two primary portions are connected. One of the portions has a channel through which the IV lumen will pass and the automatic clamp assembly is there located to bear on the lumen. The other portion of the book-like assembly houses the servo mechanism and the electronics which control the movement of the adjustable clamp. In particular, the present invention lends itself to being approximately the size of a small cassette tape player with dimensions of approximately less than 8 inches long, 5 inches wide and 2 inches deep. The total weight of the unit is less than one pound so that it is portable, and the casing in which the components are carried is shock-resistant and highly indestructible to provide a unique automatic adjustable clamp assembly easily adapted to be mounted on an IV lumen and be self-contained and battery driven without the need for exterior wires or controls. Any number of such units can be concurrently used on any number of patients with minimum nursing supervision, and the simplicity and ease of operation is unmatched.

Trauma, Blood Loss and Shock

Major trauma associated with military combat, accidents and massive civilian casualties is always associated with hemorrhage, blood loss and shock. The inadequate perfusion of major organs by the circulatory system will soon lead to death of the patient unless there is rapid replacement of the fluid that has been lost. The severity of the loss (shock) and the efficacy of the replacement therapy is measured by the levels and changes in the patient's measurable blood pressure. The optimal course involves the rapid infusion of IV fluids, frequently accompanied by assistive drugs (vasopressors) that serve to bring the patient's pressure to normal levels and keep it there as the intravenous infusion continues. The overloading of the circulatory system with IV fluids (flooding) is equally dangerous and must also be avoided.

This device and the mechanical controller will work in a manner similar to that in my prior patent applications, by reacting to pre-set values in Blood Pressure; controlling and monitoring fluid flow response until the patient's pressure reaches normal levels; controlling flow rate to maintain those normal levels; and assuring that there will never be an excess of fluid delivered intravenously. This will be accomplished automatically by the closed loop system in response to the patient's own Blood Pressure values, as determined by continuous and serial automatic reading of the monitoring apparatus.

The same versatility of the device applies in the situation of Acute Blood Loss Shock Syndrome. Such benefits and features include accurate therapeutic response; simplicity; safety; portability; adaptability for field use and many emergency clinical situations; automated treatment by a closed loop system allowing control of multiple patients safely and effectively by a single qualified care giver.

The device is uniquely suited and adaptable for field use in the non-hospital environment. However its simplicity, portability, low cost and automated physiologic response capabilities will also permit it to be used effectively within the hospital environment. Multiple units in an intensive care setting would be an excellent application of this mobile automated technology, providing back-up safety mechanisms and relieving the professional staff of some of its labor intensive patient monitoring responsibilities.

### BRIEF DESCRIPTION OF THE DRAWINGS

FIG. 1 is a pictorial view of the entire assembly of this invention for a single patient.

FIG. 2 is a preferred embodiment of the housing of this invention shown with two portions hingedly connected having a channel through which the lumen passes.

FIG. 3 is a breakaway view of the invention shown in FIG. 2 with the major components there illustrated.

FIG. 4 is yet another breakaway view of the invention showing only so much of the assembly as required during operation.

FIG. 5 is an illustrative mechanical means showing the adjustable clamp assembly bearing on the lumen causing the lumen to adjust its cross-sectional area.

FIG. 6 is an alternate embodiment of that shown in FIG. 5.

### DETAILED DESCRIPTION OF THE PREFERRED EMBODIMENT

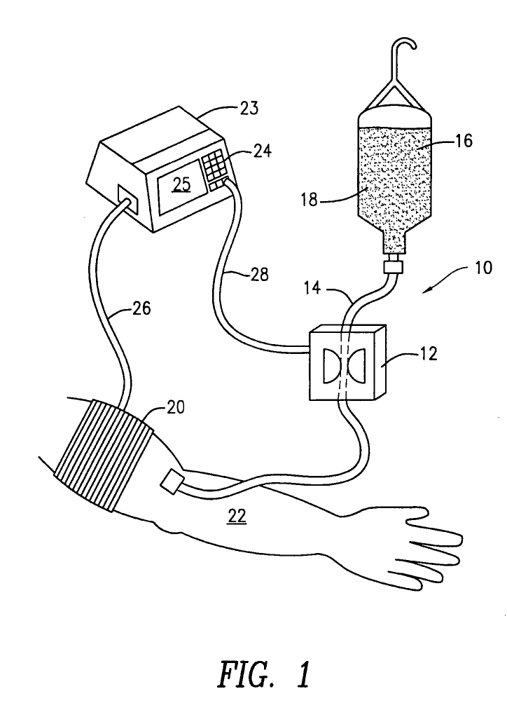

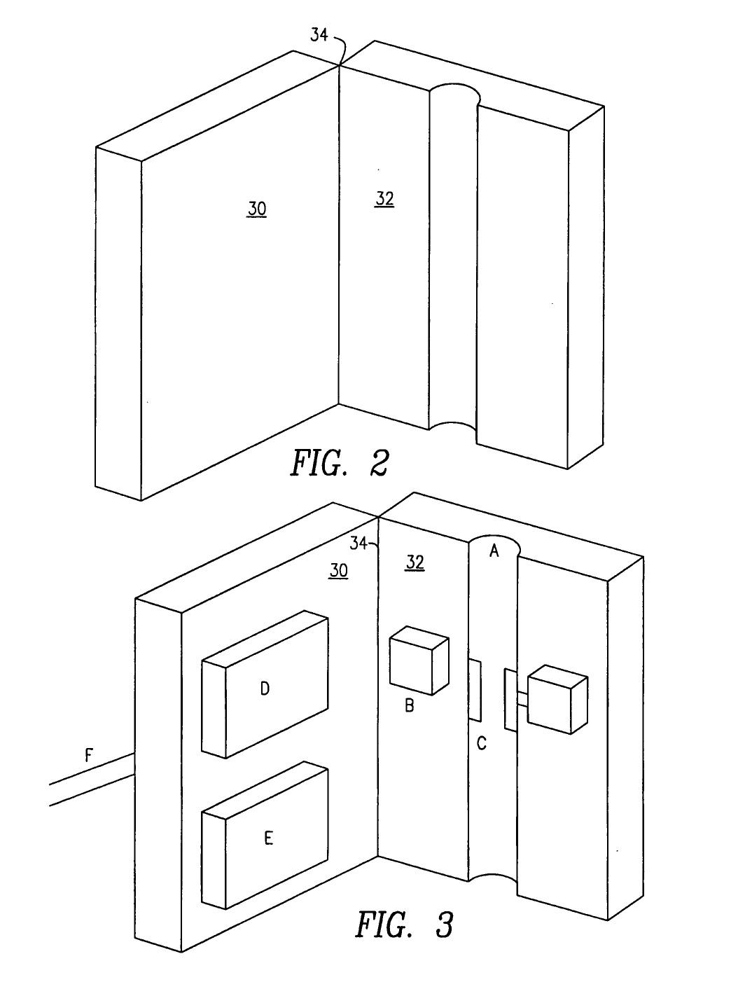

FIG. 1 is a view somewhat similar to a single station patient automatic control monitor as illustrated in FIG. 2 of U.S. Pat. No. 6,165,151. That system relates to sensing oxygen in the blood with a pulse oximeter and adjusting fluid flow to a patient in order to achieve a desired oxygen level in the blood of the patient. A single station assembly is identified, and mechanical control of the cross-sectional area of the IV lumen is identified as controlled in the '151 patent.

The present invention is directed to control of shock and maintenance of blood pressure following trauma or severe blood loss. Many patients may be automatically monitored with minimum medical supervision in order to deal with a plurality of serious patient conditions concurrently in a safe, effective and efficient manner following trauma.

The need for large scale field location medical administration is increasingly apparent in today's environment. Natural and other man-caused mass calamities require immediate medical supervision of large numbers of patients suffering serious trauma. Because of the possibility of large scale casualties requiring concurrent supervision and administration, the present invention will lend itself to effective and widespread use. In particular, the present invention presents a system to monitor real time patient blood pressure and automatically control fluid flow to the patient to maintain and sustain a desired blood pressure level.

A conventional IV gravity feed system 10 is employed, and the present invention merely alters such conventional IV system by utilizing an adjustable clamp assembly 12 adapted to sit on an IV lumen 14, which clamp is controlled to control the cross-sectional size of the lumen thereby to control the quantity of fluid supplied to a patient.

The adjustable clamp assembly 12 (shown in more detail below) employs low power consumption by utilizing micro-motors battery operated which are responsive to electrical signals. The system of FIG. 1 illustrate an IV bag or reservoir 16 illustrated as being hung in a conventional fashion so that gravity feed enable the fluid 18 contained therein to drip into the patient's arm, as illustrated. A blood pressure collar 20 is wrapped around the arm 22, and the blood pressure of the patient is determined in a conventional automatic fashion by an automatic controller 24 which is controlled to periodically inflate collar 20 through conduit 26 which conduit also carries electrical wires to carry the blood pressure reading to controller 23. In one elementary embodiment controller 23 includes a key pad 24 and a display 25 with key pad 24 used for entering desired physiological data which may be preset for the patient. The controller 23 includes a conventional data processing digital system to compare the real-time measured blood pressure to a pre-set desired blood pressure level and to generate an “error” signal along electrical conductor 28 which then controls the adjustable clamp assembly to control the size of the cross-sectional opening to increase, decrease or leave unchanged fluid 18 being supplied to the patient until the pre-set level is reached. By periodically initiating the blood pressure measurement, the patient blood level can be maintained at a desired level.

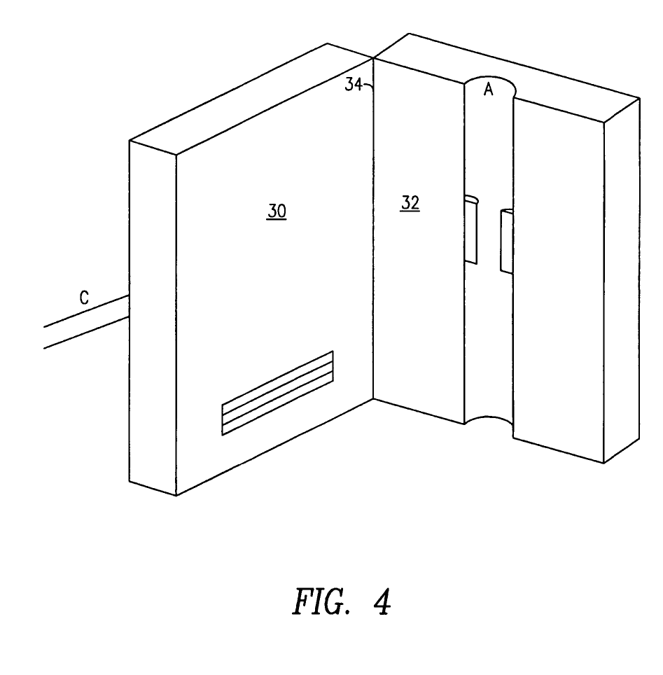

FIG. 2 is a perspective view of an illustrative embodiment of the housing of this invention. As shown, the housing comprises two primary sections 30 and 32 connected by a hinge 34 as like a book having front and back halves 30 and 32. FIG. 2 presents the two sections as though the facing faces are plain, although there will be more seen in the front face in ordinary use as illustrated in FIG. 4. In any case, FIG. 2 illustrates that this is a very compact unit in which the servo control, clamps, electronics and all are housed in a very compact unit and in which a channel is provided which contacts on the lumen when the two-part assembly is closed about hinge 34 thereby holding the lumen and the invention together.

FIG. 3 is a breakaway view of that shown in FIG. 2 and illustrates the major components of the invention operatively shown in FIG. 1. As stated above, this device is small, compact, lightweight, shock resistant and very durable. As such, it can be readily utilized in any field environment and many of such items can be concurrently used with one per patient so as to ensure automated monitoring of patient blood pressure during high trauma situations. Additionally, such devices may lend themselves to widespread use in hospital environments because of the compact nature of the construction, its simplicity of operation, automatic control and the minimization of the need for nursing supervision.

As illustrated in FIG. 3, reference numeral A refers to and shows a longitudinal channel running from top to bottom in which the lumen will be inserted. Reference letter B illustrates in block form the clamp mechanism and controller which moves clamps C to compress or otherwise move to alter the cross-sectional size of the lumen passing through this invention. Reference letter D illustrates the location for the power supply which will be battery operated, and reference numeral E illustrates the servo mechanism where the associated electronics is located for comparing the real-time current patient blood pressure level with a pre-set level which is that which is necessary to maintain the patient from going into sever trauma induced problems. Reference letter F shows the tubular connection which goes to the patient collar 20 shown in FIG. 1 which carries blood pressure information as well as administering the blood pressure test. While a collar is shown in FIG. 1, more simplistic and current modern blood pressure sensitive measurements devices may be utilized which also lends to the additional light weight of the present invention.

FIG. 4 is yet another view of the present invention which reference letter F identifies the control range for the IV so that a display of the current pressure or set desired blood pressure can be easily determined and set.

As may easily be understood, when the two part assembly of the invention shown in FIGS. 2-4 are closed about the IV lumen, a close fit is thereby maintained ensuring that the assembly of the present invention closely sits on and is firmly held to the lumen so as to ensure that the operation of the adjustable clamps C is effectively maintained in order to achieve the desirable change in cross-sectional area thereby controlling the amount of fluid flow to the patient.

In some cases, assistive drugs such as vasopressors are required, and the assistive drugs will be premixed in the standard IV solution.

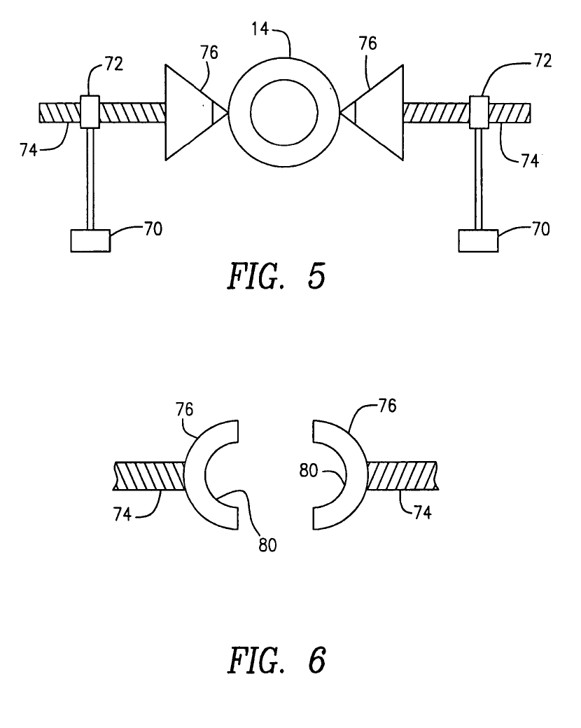

FIGS. 5 and 6 illustrate one preferred embodiment for an adjustable clamp. In particular, micromotors 70 are provided which receive the control signals to control the movement of the adjustable clamp assembly 12 which includes a rotatable threaded collar 72 through which a threaded rod 74 passes. One end of threaded rod 74 terminates in a compressor block 76, and a pair of such compressor block assemblies 76 are illustrated in FIG. 5, which bear against lumen 14 to control the cross-sectional size of the lumen thereby controlling the rate of fluid flow to the patient. Micromotors 10 are battery operated miniaturized micromotors which are moved only when a control signal is present, thereby reducing power drain. Such micromotors are especially well adapted for field environments in which multiple trauma patients are concurrently being treated without the requirement for individual medical personnel at each and every patient station.

FIG. 6 is an alternative embodiment of a split collar assembly 78 having oppositely disposed arcuate members 80 which bear upon IV lumen 14 more effectively controlling the cross-sectional size of the lumen than the pressure blocks 76 illustrated in FIG. 5. The arcuate members 80 are mounted facing to one end each of threaded rods 74.

It should be understood that the preferred embodiment was described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly legally and equitably entitled.

1. A system for automatically monitoring a patient's blood pressure level and controlling fluid flow to said patient, said system comprising:
a conventional gravity feed fluid flow system to infuse fluid into a patient,
said conventional feed fluid flow system comprising a container holding said fluid at a level higher than the point of infusion of said liquid into said patient,
an IV tube lumen connected between said container and said patient,
an adjustable clamp assembly attached to said IV lumen and having physical abutment means to bear on said IV lumen to control the cross-sectional size of said IV lumen to control the rate of fluid flow through said IV lumen,
a blood pressure measurement device attached to said patient to provide a signal conveying the real-time blood pressure level of said patient,
a data processor connected to receive said signal, said data processor comprising pre-set blood pressure values reflecting the state of a patient's desired condition and means to compare said signal to said pre-set blood pressure values to generate a control signal,
said control signal reflecting the difference between said signal and said desired level for the pre-set blood pressure value and being supplied to said adjustable clamp assembly, and
said adjustable clamp assembly comprising a battery operated micromotor controlling said adjustable clamp responsive to said control signal to control the rate of fluid flow to said patient.

2. A system as set forth in claim 1, wherein a blood pressure measurement device comprises a conventional blood pressure collar mounted on the arm of the patient.

3. A system as set forth in claim 1, wherein said adjustable clamp is housed within a shock resistant assembly.

4. A system as set forth in claim 1, wherein said system is housed in a housing, said housing having a channel extending vertically, the channel being sized to hold onto said IV lumen for the length of said channel.

5. A system as set forth in claim 4, wherein said housing comprises a two-part assembly hingedly connected together, said channel extending vertically therethrough, said housing causing said channel to contact said IV lumen when said two part assembly enclosed about said hinge.

6. A system as set forth in claim 4, wherein said system weighs less than a pound.

7. A system as set forth in claim 5, wherein said housing weighs less than a pound.

8. A system as set forth in claim 1, wherein said adjustable clamp comprises a portable battery operated micromotor.

9. A system as set forth in claim 4, wherein said housing comprises a book-like structure having two rectangular portions, a front and a back with the front hingedly connected to the back and said channel formed vertically through the entirety of said housing.

10. A system as set forth in claim 4, wherein said housing is less than eight inches long by five inches wide and two inches thick and said channel is in the same direction as the length.

11. A system as set forth in claim 10, wherein said system weighs less than one pound.
